(19) United States
(12) Patent Application Publication
(10) Pub. No.: US 2020/0375943 A1
(43) Pub. Date: Dec. 3, 2020

(54) CYTOCIDAL METHOD OF CANCER CELLS SELECTIVELY IN HUMAN PATIENTS CLINICALLY BY DEPLETION OF L-ASCORBIC ACID, PRIMARILY, WITH ITS SUPPLEMENTATION ALTERNATELY AUGMENTING ITS CYTOCIDAL EFFECT

(71) Applicant: Chan Hyung PARK, Des Moines, WA (US)

(72) Inventor: Chan Hyung PARK, Des Moines, WA (US)

(21) Appl. No.: 16/886,749

(22) Filed: May 28, 2020

Related U.S. Application Data
(60) Provisional application No. 62/856,063, filed on Jun. 1, 2019.

(51) Int. Cl. A61K 31/375; A61K 31/4045; A61K 31/472; A61K 45/06

(52) U.S. Cl. CPC A61K 31/375; A61K 45/06; A61K 31/472; A61K 31/4045

(57) ABSTRACT

A method is provided for treating a patient with a disease. The method includes administration of a repeated cycle of alternation of a rapid depletion of L-ascorbic acid (LAA) and supplementation of a high dose of LAA, wherein the rapid depletion of LAA is achieved by blocking of LAA signal transduction and/or by blocking of LAA transporter to control growth of malignant cells in a body of the patient. Further, a pharmaceutical composition designed to perform at least one of: a blocking of a LAA signal transduction and/or a blocking of a LAA transporter is provided. The pharmaceutical composition may be used as a depletion means of LAA in a cyclic administration of the depletion of LAA followed by the high dose supplementation of LAA over a period of time.

### CROSS-REFERENCES AND RELATED APPLICATIONS

This application claims priority to U.S. Provisional Application No. 62/856,063, filed Jun. 1, 2019, titled “CYTOCIDAL METHOD OF CANCER CELLS SELECTIVELY IN HUMAN PATIENTS CLINICALLY BY DEPLETION OF LAA, PRIMARILY, WITH ITS SUPPLEMENTATION ALTERNATELY AUGMENTING ITS CYTOCIDAL EFFECT,” the content of which is incorporated herein by reference in its entirety.

### TECHNICAL FIELD

The present disclosure relates to methods or techniques for mitigating or reducing the growth of malignant tumor cells selectively in human patients, in particular, by means of rapid depletion of L-ascorbic acid (LAA) with supplementation of a high dose LAA in an alternative cyclic fashion over a period of time, as well as composition or means for achieving the rapid depletion of LAA.

### BACKGROUND

Vitamin C is known as L-Ascorbic Acid (LAA) and has been widely used for health supplements for people around the world. Also, in 1971 a seminal discovery was made that L-ascorbic acid (LAA) is highly and selectively essential for survival of cancer cells (e.g., myeloma cells) which was made at a research laboratory of University of Toronto where Best and Banting discovered Insulin. Normal non-malignant, healthy cells such as granulocytes were shown to be not affected at all by presence of LAA (that is, the normal healthy cells grew well with or without LAA) but the malignant tumor cells or leukemia cells needed the presence of LAA for their growth. Over the years, this cell culture method for the growth of non-malignant or benign granulocytes or neutrophils has been proven to be clinically relevant as the Granulocyte-Colony Stimulating Factor (G-CSF) which has been widely used for the past decades up until now to mitigate against neutropenia of patients receiving cancer chemotherapy. Neutropenia is a condition of a low white blood cell count. As such, there are many pharmaceutical firms, at least 10 of them, manufacturing or selling G-CSF and there are at least two pharmaceutical firms manufacturing its biosimilars that are to be used for supportive treatment for cancer patients.

The 1971 seminal discovery had used the same cell culture system for the G-CSF which was widely used at the time for the growth of granulocytes and found that the only difference for the growth of malignant cells, compared to the one for the growth of healthy cells, is additional and absolute requirement of LAA for the growth of malignant cells. While normal healthy cells (e.g., non-malignant granulocytes) are not affected at all by the absence of LAA, i.e., growing well with or without LAA, it was observed that the absence of LAA had an absolute negative effect on the growth of malignant cells exclusively. In fact, this astonishing discovery was made by the inventor and his colleagues, which was recognized by multiple peers and investigators again as in a published article in Science journal and led to awards of NIH/NCI research supports in the form of Investigator-Initiated Grants of RO1 type for 3 times in consecutive terms (RO1 CA201717) and a research career development award (KO4 CA00534) for the inventor. Further, this seminal discovery was validated by the cell-culture studies on bone marrows from 259 patients with acute myelogenous leukemia and myelodysplastic syndromes (the latter also called pre-leukemia) was published in Cancer Research 45:3959-3973, as a journal article titled “Biologic Nature of the Effects of LAA on the Growth of Human Leukemic Cells” (Reference 3), which is incorporated herein by reference in its entirety. In this research, it was noted that close to 50% of patients had this unique requirement of LAA. We have had altogether close to 500 patients with bone marrow samples with some of these from SWOG (previously called with Southwest Oncology Group) affiliated hospitals such as the University of Michigan per SWOG protocol. It was shown that the same 50% of patients basically showed this requirement of LAA for the growth of the malignant cells whatever hospital from which the bone marrow samples came.

Since the seminal discovery on this myeloma by the inventor and his colleagues, there had been indications that LAA is also important for the growth of multiple other tumor types including not only acute myelogenous leukemia and myelodysplastic syndromes by our group, but also for acute lymphoblastic leukemia, and sarcoma by other group (see Reference 5). Also, there was indirect evidence that the approach of using depletion of LAA may help in fighting colon cancer (see Reference 6). This concept of the beneficial effect of LAA depletion followed by supplementation of a high dose LAA was recently supported by another group with animal models xenografted with human colorectal cancers (and other KRAS mutant lung and pancreatic cancers) (see Reference 50; Di Tano M, et al. Synergistic effect of fasting-mimicking diet and vitamin C against KRAS mutated cancers. Nature Communications 2020; 11:2332).

Further, not only that the positive effects of the depletion of LAA on the growth of tumors have been noted but also that the supplementation of a high dose LAA may also result in favorable effect on the growth of tumors such as prostate, pancreatic, liver, and colon cancer cells (reference 11 and www.mercola.com). So far, research efforts have focused on either the depletion of LAA alone or the supplementation of a high dose LAA alone to reduce the growth of malignant tumor or cancer cells.

Thus, there is still a need for new and improved techniques for mitigating or reducing the growth of malignant tumor or cancer cells in human subjects with little toxicities or side-effects.

### SUMMARY

In the past decades, as noted above, it was established that the depletion of LAA may be used to control the growth of multiple tumor types including not only acute myelogenous leukemia and myelodysplastic syndromes, but also multiple myeloma and other types of tumors.

Earlier, the positive effects on the growth of tumors have been noted with the depletion of LAA only. It is also noted that the supplementation of LAA may also result in favorable effect on the growth of tumors. However, all the studies have focused on either the depletion of LAA alone or the supplementation of LAA alone. The present disclosure, in an aspect, focuses on the inter-dependency between the depletion of LAA and the supplementation of LAA and thus the cyclic alternation of the depletion of LAA and the supplementation of LAA over a period of time.

In an aspect of the present disclosure, the inter-dependency of the depletion of LAA and the supplementation of LAA may be utilized as a form of an effective treatment method for reducing or combatting the growth of tumor or cancer cells with little toxicities.

According to the present disclosure, a method for treating a patient with a disease or a certain type of malignant tumor is provided. By way of example, the method includes administration of a repeated cycle of alternation of a rapid depletion of LAA and supplementation of a high dose of LAA in a body of a patient over a period of time. The rapid depletion of LAA may be achieved by blocking of LAA signal transduction and/or by blocking of LAA transporter to control growth of malignant cells in the body of the patient.

In an aspect of the present disclosure, the rapid depletion of LAA may be accomplished by blocking its signal transduction or transporter, and its supplementation of a high dose LAA may be accomplished through an intravenous infusion method.

In another aspect of the present disclosure, the disease (malignancies) may be cancer comprising myeloma, acute myelogenous leukemia, myelodysplastic syndromes, acute lymphoblastic leukemia, sarcoma, non-small cell lung cancer, melanoma, oral, gastrointestinal, colon, pancreatic, breast, or prostate cancer.

In another aspect of the present disclosure, the supplementation of the high dose LAA may be promptly followed within a predetermined time after the rapid depletion of LAA.

In another aspect of the present disclosure, the blocking of LAA signal transduction may be performed by a composition or drug including at least one of GF09203X or H-89.

In another aspect of the present disclosure, the blocking of LAA signal transporter may be performed by a composition or drug including at least one of: a short form of human SVCT2 (hSVCT2-short), flavonoid phloretin, quercetin, myricetin, genistein, a non-steroidal anti-inflammatory drug (NSAID) including indomethacin and/or diclofenac, or linsidomine.

In an aspect of the present technology, a pharmaceutical composition is provided for inducing a rapid depletion of LAA in a body of a patient to reduce or mitigate growth of malignant tumor or cancer cells in the body. The pharmaceutical composition may include a composition designed to perform at least one of: a blocking of a LAA signal transduction and/or a blocking of a LAA transporter. In an aspect of the present disclosure, the disease may include a cancer and wherein the cancer comprises myeloma, acute myelogenous leukemia, myelodysplastic syndrome, acute lymphoblastic leukemia, sarcoma, breast, non-small cell lung cancer, melanoma, oral, gastrointestinal, colon, pancreatic or prostate cancer. Further, the composition may include at least one of: GF109203X or H-89. Furthermore, the composition may include at least one of: a short form of human SVCT2 (hSVCT2-short), flavonoid phloretin, quercetin, myricetin, genistein, a non-steroidal anti-inflammatory drug (NSAID) including indomethacin and/or diclofenac, or linsidomine.

### BRIEF DESCRIPTION OF THE DRAWINGS

These and other features, aspects and advantages of the present disclosure will become better understood from the following description, appended claims, and accompanying figures where:

FIGS. 1A and 1B are example block diagrams conceptually illustrating one or more aspects of the present disclosure;

FIGS. 2A and 2B are example graphical representations illustrating effects of the present technology in accordance with an aspect of the present disclosure;

FIGS. 3A and 3B are example graphical representations illustrating effects of the present technology in accordance with an aspect of the present disclosure;

FIGS. 4A and 4B are example block diagrams conceptually illustrating one or more aspects of the present disclosure;

FIGS. 5A, 5B, and 5C are example graphic and tabular data conceptually illustrating one or more aspects of the present disclosure;

FIG. 6 is example graphical data conceptually illustrating one or more aspects of the present disclosure;

FIGS. 7A and 7B are example block diagrams conceptually illustrating one or more aspects of the present disclosure;

FIG. 8 is a block diagram conceptually illustrating one or more aspects of the present disclosure;

FIG. 9 is a diagram conceptually illustrating one or more aspects of the present disclosure; and

FIG. 10 is a diagram conceptually illustrating one or more aspects of the present disclosure.

### DETAILED DESCRIPTION OF THE EMBODIMENTS

The detailed description of illustrative examples will now be set forth below in connection with the various drawings. The description below is intended to be exemplary and in no way limit the scope of the present technology. It provides a detailed example of possible implementation and is not intended to represent the only configuration in which the concepts described herein may be practiced. As such, the detailed description includes specific details for the purpose of providing a thorough understanding of various concepts, and it is noted that these concepts may be practiced without these specific details. In some instances, well known structures and components are shown in block diagram form in order to avoid obscuring such concepts. It is noted that like reference numerals are used in the drawings to denote like elements and features.

Further, methods and devices that implement example embodiments of various features of the present technology are described herein. Reference in the description herein to “one embodiment” or “an embodiment” is intended to indicate that a particular feature, structure, or characteristic described in connection with the example embodiments is included in at least an embodiment of the present technology or disclosure. The phrases “in one embodiment” or “an embodiment” in various places in the description herein are not necessarily all referring to the same embodiment.

In the following description, specific details are given to provide a thorough understanding of the example embodiments. However, it will be understood by one of ordinary skills in the art that the example embodiments may be practiced without these specific details. Well-known structures and techniques may not be shown in detail in order not to obscure the example embodiments.

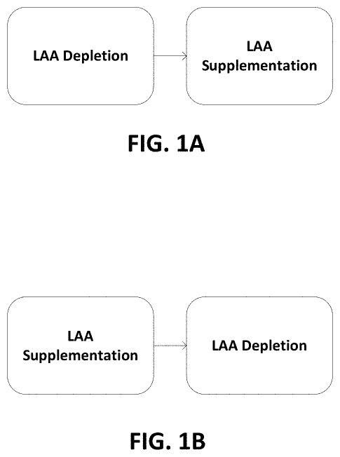

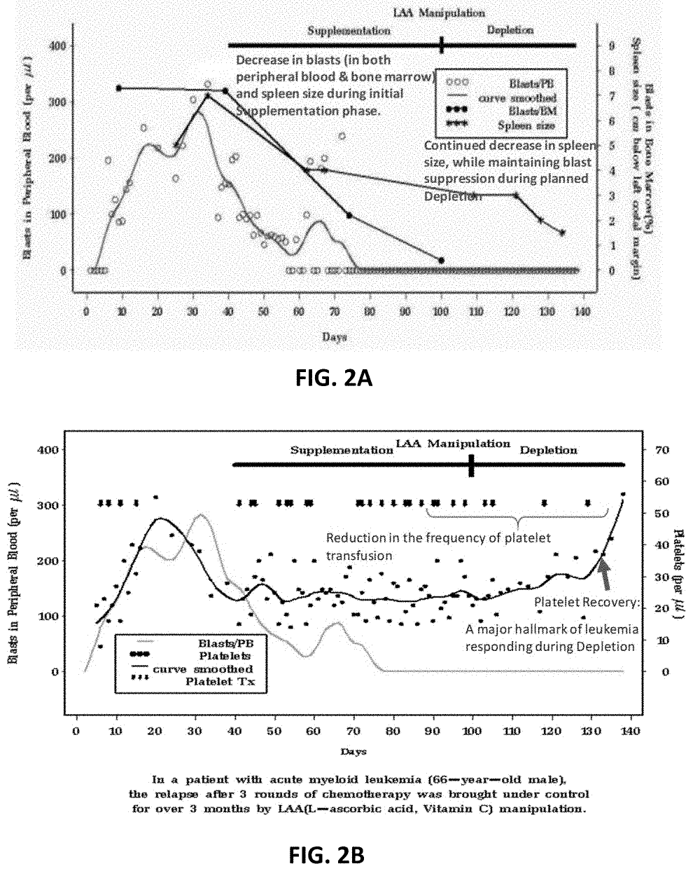

The present disclosure provides a new concept of utilizing the interdependence between depletion of L-ascorbic acid (LAA) and supplementation of LAA as an effective treatment method for reducing or combatting the growth of malignant tumor or cancer cells in a human patient. In an aspect of the present disclosure, as shown in FIGS. 1A and 1B, the depletion of LAA followed by its supplementation may be more effective in combating cancer cells, than the depletion of LAA alone. Alternatively, the supplementation of LAA followed by the depletion of LAA may be more effective than the supplementation of LAA alone. As shown in FIGS. 2-5, the present disclosure provides an alternation of rapid depletion and supplementation of a high dose LAA infusion in a controlled manner, e.g., a cyclic administration of LAA manipulation over a period of time.

When it comes to the growth of cells, it has been shown that there are essential molecules for the growth of cells. In particular, over the decades, it has been observed that granulocyte-colony stimulating factor (G-CSF) has been shown to be essential for the growth of normal white blood cells and erythropoietin (EPO) is essential for the growth of normal red blood cells. Also, researchers including the inventor and others in the past demonstrated that LAA is essential for the growth of malignant white blood cells, for example, leukemic cells (see References 1-4).

G-CSF is a growth factor that is used to stimulate the production of granulocytes, i.e., a type of white blood cells, and as such, it may be used as a drug in patients undergoing therapy that will cause low white blood cell counts (i.e., neutropenia). G-CSF stimulates the bone marrow (which is a spongy material inside bones) to make while blood cells. As such, over the years it has been used as a support medication for many patients. In particular, G-CSF can be given to a patient via subcutaneous injection (injection in the layer between the skin and muscle) or intravenous infusion (infusion into a vein). G-CSF has been used over decades to mitigate neutropenia following chemotherapy indirectly helping cancer treatment. G-CSF drug manufacturers include Cadila Pharmaceuticals, Abbott Laboratories and many others.

Erythropoietin is a positive regulator of erythropoiesis that facilitates proliferation of red blood cells that deliver oxygen to the whole body of a person. It is a hormone produced by the kidney that is critical in the formation of red blood cells. Erythropoietin stimulating agents are used often for patients with long term kidney disease and anemia.

L-ascorbic acid (LAA) is a chemically active form of Vitamin C and a well-known antioxidant having many beneficial health effects. Further, as mentioned herein, it is also documented that malignant tumor cells need LAA for the growth of the malignant tumor cells. In a journal article titled, “Ascorbic acid: A culture requirement for colony formation by mouse plasmacytoma cells” by CH Park et al, Science 12 Nov. 1971, Vol. 172, Issue 4010 (see Reference 1), which is incorporated herein by reference in its entirety, the inventor and his team established that while normal counterpart hemopoietic cell is not influenced at all by the presence of LAA, e.g., healthy cells growing well with or without it, the malignant tumor cells, e.g., a mouse myeloma and human leukemic cells needed the presence of LAA for their growth. It is known that the LAA is essential for the continued growth of malignant tumor cells and thus the control of LAA may lead to an effective fighting mechanism against the growth of malignant tumor or cancer cells in a patient.

### Cyclic Manipulation of LAA in a Controlled Manner

By way of example, FIGS. 2A and 2B illustrate the effect of supplementation of LAA followed by the depletion of LAA on leukemic blast cells (see Reference 7), based on unpublished clinical data. As shown in FIG. 2A, the high dose supplementation of LAA was followed by the depletion of LAA after 100 days. In the example, for the depletion of LAA, patients were placed on a diet deficient in LAA and for the supplementation of LAA, the patients received intravenous administration of LAA according to the protocol of Hoffer L J, Levine M et al. Ann Oncol 2008 (Reference 12). As can be seen in FIG. 2A, during the supplementation of LAA phase, a decline in leukemic blast cell number (in both peripheral blood and bone marrow) as well as a decline in spleen size are observed. Further, during the depletion of LAA phase which followed the supplementation of LAA, a continued decline in the spleen size is observed while maintaining the blast suppression.

Further, FIG. 2B illustrates additional data on the same patient as in FIG. 2A and shows a marked reduction in the frequency of platelet transfusion with more rapid platelet recovery, thereby resulting in the near complete cessation of platelet transfusion. This provides a major indication that leukemia is responding to the manipulation of LAA administration, e.g., alternation of LAA supplementation and LAA depletion. In fact, as shown in FIG. 2B, the recovery or elevation of platelet counts during the depletion phase of LAA with the near complete cessation of platelet transfusion is a major hallmark of leukemia response while the spleen size continued to shrink, according to an article titled “Report of an international working group to standardize response criteria for myelodysplastic syndromes” by Bruce D. Cheson, et al. Vol. 96. No. 12, Blood. 2000 (see Reference 9). Further, it has been shown that a patient started with depletion of LAA first exhibited favorable signs and ensuring supplementation showed positive benefits. As such, FIGS. 2A and 2B demonstrates the significant values of having both the depletion of LAA and the supplementation of LAA.

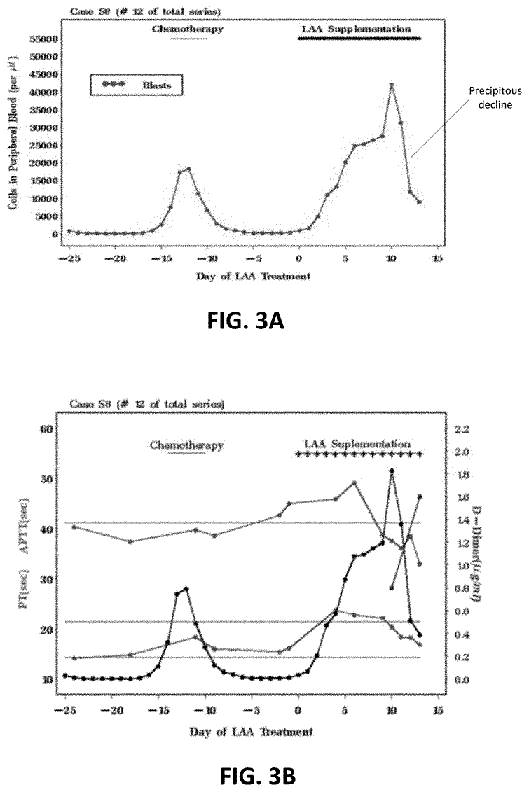

In fact, in case of leukemia, the therapeutic effect of LAA manipulation is clearly observed as shown in FIGS. 3A and 3B. FIG. 3A shows leukemia blasts in peripheral blood observed over a treatment period with the LAA supplementation following a cytotoxic chemotherapy. That is, in the example, a leukemia patient first received a chemotherapy and subsequently received the LAA supplementation. As shown in FIG. 3A, in the case of leukemia, the patient exhibited relapse with rapidly rising blasts in 10 days after the last dose of cytotoxic chemotherapy. However, after another 10 days of the LAA supplementation, a sharp decline (e.g., a precipitous decline) in the leukemic blast cells after a short rise was observed too. The noted sharp decline is much more precipitous than what can be observed with chemotherapy. As such, the relapse after prior chemotherapy so soon is a highly ominous situation, and yet with the high dose of the supplementation of LAA in the state of LAA deficiency (due to moribund state with poor food intake after cytotoxic chemotherapy), the precipitous decline of leukemia blast cells is quiet striking. Thus, it may be concluded that the therapeutic effect of LAA occurs only after a period of LAA deficiency.

Additionally, it is noted that a well-known D-dimer test for massive cell lysis, such as shown in mis-matched transfusion, showed a sharp increase in support of this massive lysis of leukemic cells, as shown in FIG. 3B, further substantiating the rapid cell lysis of a large number of leukemic cells in the blood. In the example, the significant improvement is also noted that there was a total of 8 out of 16 patients in the Phase I/II clinical trial responding (p=0.007). A new in-depth examination and analysis of data acquired in the phase I/II clinical trials (IRB approved, registered at US government site, ClinicalTrials.gov identifier: NCT00329498) on 16 evaluated patients (out of total 18 patients enrolled) reveals a statistically significant outcome as shown below in Table 1.

TABLE 1
Clinical Responses Predicted by In-Vitro Assay
Clinical Responses Predicted by in Vitro Assay

| | Clinical Responses | | |
|---|---|---|---|
| In Vitro Assay | Yes | No | Total |
| Sensitive | [symbol] | [symbol] | 9 |
| Non-sensitive | [symbol] | [symbol] | 6 |
| Indeterminate | 1 | 0 | 1 |
| Total | 8 | 8 | 16 |

In Table 1 above, the response rate of selected (in vitro sensitive) patients is 7 out of 9 (or 78%) which leads to a highly significant value with p=0.007 by Fisher's Exact Test (2×2 data in bold italic). As such, in an aspect of the present disclosure, it is evident that the manipulation of LAA, e.g., the depletion of LAA and its supplementation in a cyclic pattern over a period of time, may result in desired effects on killing or markedly suppressing malignant tumor or cancer cells in subject patients.

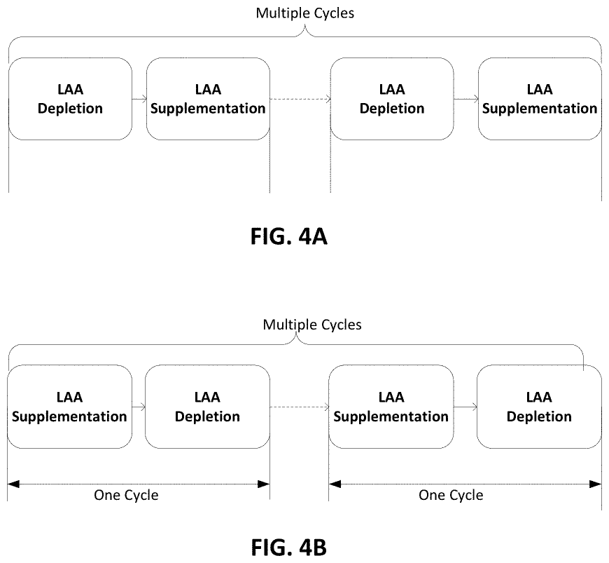

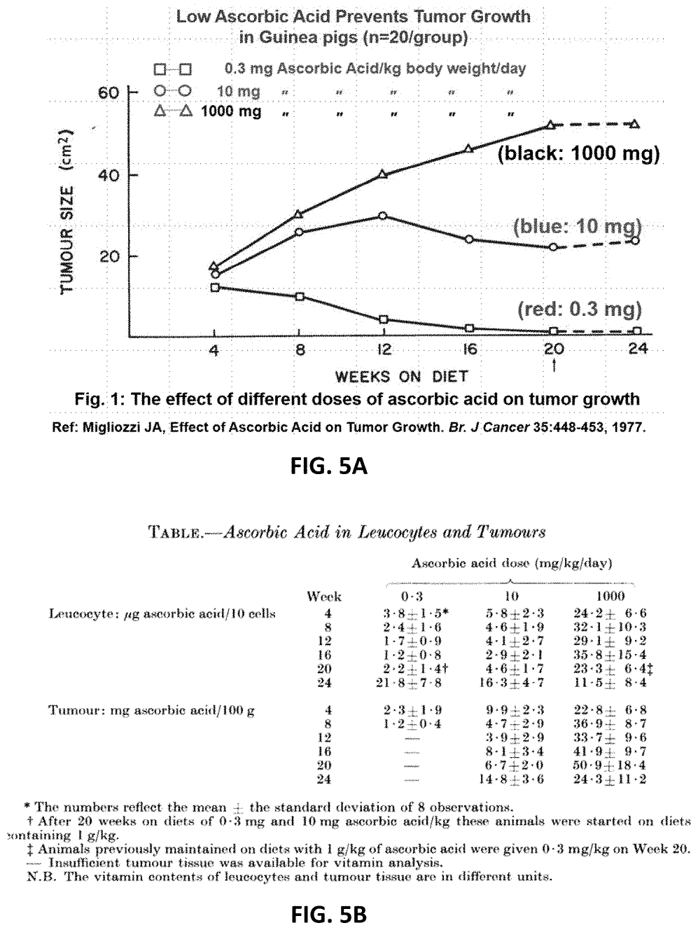

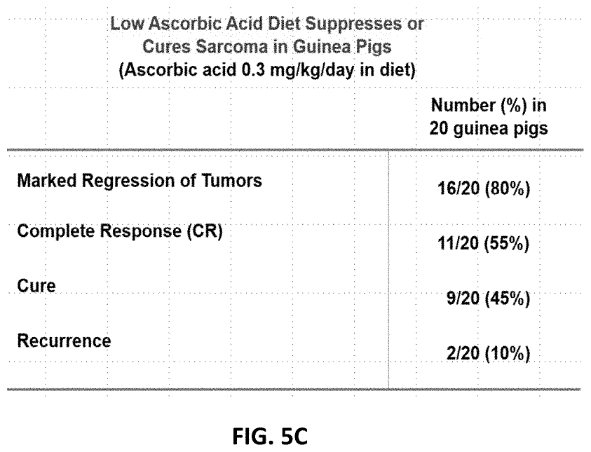

In an aspect of the present disclosure, as shown in FIGS. 4A and 4B, the manipulation of LAA may be designed and implemented as a therapeutic treatment for controlling the growth of malignant tumor or cancer cells. In one example, as shown in FIG. 4A, the therapeutic treatment may be designed such that the depletion of LAA is first administered and then the supplementation of LAA is administered in multiple cyclic times over a period of time. One cycle includes the depletion of LAA first and then the supplementation of LAA, or vice versa. In the example, as shown in FIG. 4A or 4B, the supplementation of LAA may be considered as an adjunct of the depletion of LAA. As shown in FIG. 4B, an alternating cycle of the supplementation of LAA and the depletion of LAA may be designed and administered over a period of time, in a controlled manner. Many reports showed that high doses of LAA or vitamin C suppress the growth of malignant cells and reduce the tumor volume or size. In addition, the beneficial effects of the LAA depletion or low levels of LAA were reported by another group or study titled “Effect of Ascorbic Acid on Tumor Growth” by Migliozzi J A; see Reference 52, which is incorporated herein by reference in its entirety. Migliozzi observed a significant decline in the growth of tumor in guinea-pigs, that cannot synthesize vitamin C, when they were exposed to various doses of vitamin C for over 20 weeks, as shown in FIG. 5. FIG. 5A is an annotated version of FIG. 1 of Reference 52. In the study, 84 male guinea pigs were treated with a chemical carcinogen (20-methyl-cholanthrene) and 68 of them developed sarcoma tumors, e.g., fibro-sarcomata and liposarcomata, within 140 and 180 days. Then, the author selected 60 guinea pigs and then exposed to three different doses of vitamin C (n=20 per group) via oral gavage administration: a low (0.3 mg/kg/day), medium (10 mg/kg/day), or high amount (1000 mg/kg/day) for up to additional 24 weeks. The author then measured the size of tumor via biopsy in guinea pigs in different groups.

As shown in FIG. 5A and FIG. 5B, complete tumor regression occurred in 55% of those animals receiving 0.3 mg/kg/day ascorbic acid, whereas animals given 10 mg/kg/day showed tumor inhibition but no regression, while tumors in animals maintained on 1 g/kg/day ascorbic acid grew without sign of retardation. Further, FIG. 5B (which is Table 1 of Reference 52) shows the total ascorbic acid concentrations in leucocytes and tumors, corroborating that ascorbic acid is an indispensable requirement for the growth of tumor cells. The study results by Migliozzi may be summarized in FIG. 5C, evidencing that low ascorbic acid concentration suppresses or cures sarcoma in guinea pigs.

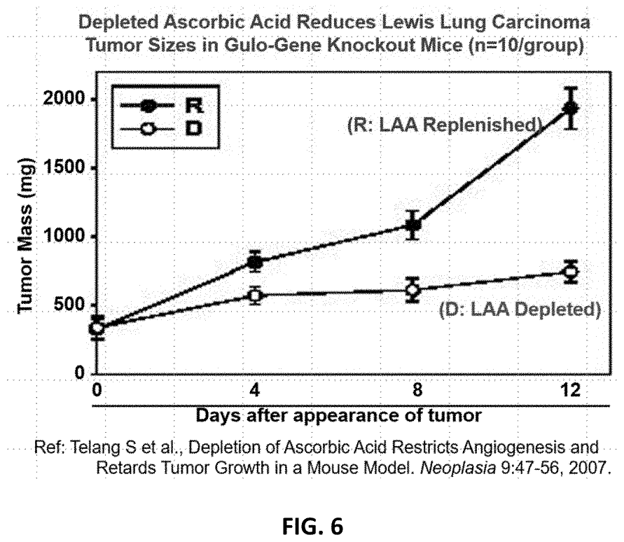

Furthermore, another recent study showed that depleted ascorbic acid reduced the size of Lewis Lung Carcinoma tumor in mice incapable of synthesizing ascorbic acid through genetic deletion of a protein (enzyme L-gulono-γ-lactone oxidase) needed for vitamin C synthesis. See a journal article titled “Depletion of Ascorbic Acid Restricts Angiogenesis and Retards Tumor Growth in a Mouse Model” by S. Telang, et al. Neoplasia 9:47-56, 2007 (Reference 53), which is incorporated herein by reference in its entirety. As shown in FIG. 6 (which is an annotated version of FIG. 2C of Reference 53), in a single mouse model with a tumor, e.g., Lewis Lung carcinoma cells, the ascorbic acid depletion (via controlled LAA supplementation in drinking water) dramatically restricts the growth of Lewis Lung carcinoma cells in vivo. These experimental results clearly provide the proof of concept of the present disclosure of the benefits of the cyclic LAA depletion and supplementation in effective cancer treatment. Nonetheless, the most appropriate cycle of supplementation and depletion of LAA will eventually be found by “trials and errors” depending on certain types of malignant cells as well as patient profiles.

However, in one example, the most reasonable duration of depletion/or supplementation cycle may be to start with 2 weeks each, as malignant cells die and disappear completely during 2 weeks of culture period without vitamin C/or survive and proliferate forming visible colonies of multiple malignant cells during again 2 weeks. Also, the dose of LAA supplementation may be 60 g/m² qd (see Reference 12).

In another example, it may be possible to design a protocol or methodology as a therapeutic approach in such a way that the rapid depletion of LAA is followed by the high dose supplementation of LAA in the form of cyclic manipulation of LAA. Alternatively, the high dose supplementation of LAA may be followed by the rapid depletion of LAA. As mentioned, FIGS. 4A and 4B may illustrate an exemplary controlled manipulation of LAA in the form of a cyclic administration of the depletion of LAA followed by the supplementation of LAA and vice versa, to combat the growth of malignant tumor cells or cancer cells with little cellular toxicities. This may result in mutual enhancements of favorable effects of the depletion of LAA and the supplementation of LAA in cycles.

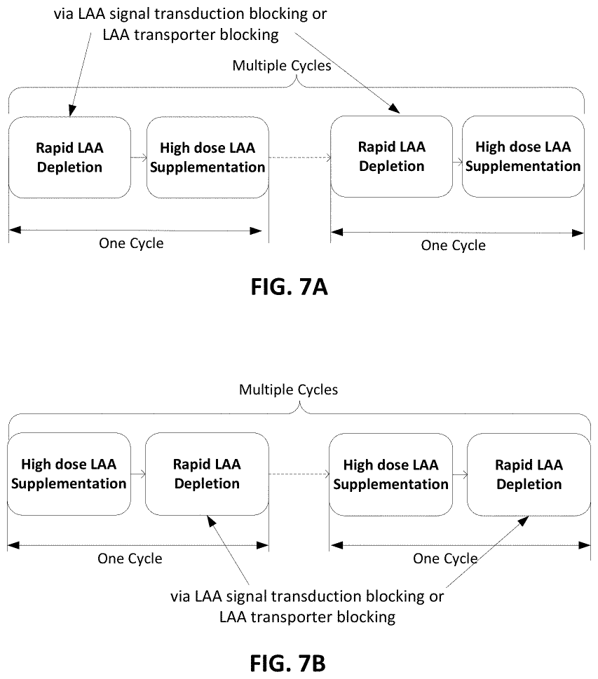

FIGS. 7A and 7B illustrate another exemplary manipulation of LAA in the form of a cyclic administration of the rapid depletion of LAA followed by the high dose supplementation of LAA or vice versa, with advanced techniques in molecular biological science to combat or mitigate the growth of malignant tumor cells or cancer cells.

So far, for the supplementation of LAA and the depletion of LAA, dietary methods have been used. However, with the dietary methods, it is not easy at all to achieve the controlled manipulation of LAA in the patients. As such, in one or more aspects of the present disclosure, advanced techniques for achieving the supplementation of a high dose LAA and the rapid depletion of LAA are disclosed herein.

Advanced Techniques for Achieving Supplementation of a High Dose LAA

The supplementation of a high dose LAA may be tried in various ways. By way of example, dietary or oral supplements may be used. However, the oral dietary supplements have limitations in terms of the intake. That is, even by taking up to 180 gram per day orally in divided doses throughout the day, the blood levels do not change and do not exceed 0.2 mMol/L because of the body's tight control. As such, because of this limitation, intravenous infusion is used because intravenous (IV) infusion bypasses the tight control of the body and results in a higher blood level with a small amount of injection. In one example, an injection of 5 gram of LAA produces a blood value of 3 mMol/L (Reference 12). Some studies suggest that malignant tumors may be killed with values between 0.5 to 3 mMol/L. That is, at mMol concentrations, LAA may be toxic to the malignant tumor cells in vitro. Further, the LAA may be given by intravenous infusion up to peak concentrations over 10 mMol without significant adverse effects to the recipient, which is two orders of magnitude above what is observed with oral supplementation. As such, the prompt supplementation of LAA is relatively easy as it can be given to a person in an intravenous manner. However, in contrast, prompt depletion of LAA has not been the case and is a very difficult task using the existing methods. Based on the necessity, we have described several methods to achieve rapid depletion of LAA in this application, as follows.

Advanced Techniques for Achieving Rapid Depletion of LAA

In an aspect of the present disclosure, at least two molecular methods may be used to achieve the prompt or rapid depletion of LAA. Based on recent advances in sciences, the blocking of signal transduction of LAA and the blocking of transporter of LAA (e.g., entry of LAA into cells) may be used to achieve the rapid depletion of LAA. In particular, as noted herein, the sodium-dependent vitamin C transporter (SVCT) may be blocked to accomplish the rapid depletion of LAA for implementation of various aspects of the present disclosure.

Although certain dietary methods may be used for the depletion of LAA, these dietary methods are not effective and not convenient to administer to a person in a controlled manner. For example, the depletion of LAA is difficult to implement to patients because the restricted diet is often hard to endure and thereby often results in the compromise of the quality of life for the patients. As such, the rapid depletion of LAA is not an easy task using the traditional methods.

In an aspect of the present disclosure, however, the present disclosure provides various example novel methods for achieving the rapid depletion of LAA which may be used alone or in combination with the high dose supplementation of LAA.

By way of example, in an aspect of the present disclosure, the rapid depletion of LAA in the body of a patient may be accomplished by an intravenous administration of an enzyme, ascorbate oxidase, as described in U.S. Pat. No. 6,989,143 B1 and EU Patent 1278536 B1, which are incorporated herein in their entirety. However, using the enzyme may not be efficient as desired.

In another aspect of the present disclosure, with advances in technology, new molecular methods may be used for achieving the rapid depletion of LAA based on blocking of signal transduction and/or transporter, for example, the blocking of LAA effect on cells at a cellular level by means of blocking of either signal transduction pathway or entry transporter for LAA.

The blocking of signal transduction is often described as a process by which a chemical signal is transmitted into a cell through a series of molecular events, for example, protein phosphorylation, catalyzed by protein kinases, which results in down-stream cellular responses. Thus, the rapid depletion of LAA may be achieved by using various techniques or means configured to achieve the blocking of the signal transduction for LAA at a cellular level. For example, any means, known at present as well as in future may be used for achieving the blocking of the signal transduction for LAA.

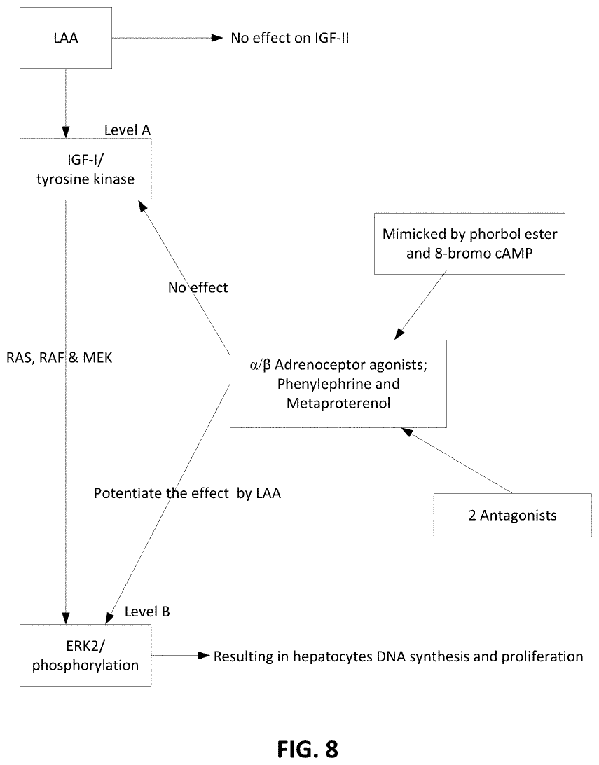

In an aspect of the present disclosure, in one example, FIG. 8 illustrates the signal transduction cascade of LAA based on research findings by Moteki et al. (See References 17 and 18), which are incorporated herein by reference in their entirety. Further, according to another study, the activation of this signal transduction for LAA in vivo has been proven to result in live cell regeneration in partially hepatectomized rats, as shown in Reference 19, which is incorporated by reference in its entirety, thereby providing in vivo relevance of this signal transduction cascade. As such, referring back to FIG. 8, the two antagonistic molecules identified by Kimura et al. (Reference 19), e.g., H-89 and GF109203X are used against the signal transduction cascade both in vitro and in vivo models of analyzing rat hepatocytes and livers. GF109203X is a direct protein kinase C (PKC) inhibitor and H-89 is a direct protein kinase A (PKA) inhibitor. GF109203X is shown to be a selective protein kinase inhibitor with the greatest effect on PKC while H-89 is shown to be a protein kinase inhibitor with the greatest effect on PKA. Both antagonist molecules are inhibitors of signal transduction for LAA.

That is, the antagonist molecules such as, e.g., H-89 and GF109203X may be used for blocking of the signal transduction for LAA to achieve the rapid depletion of LAA, thereby reducing the growth of malignant tumor or cancer cells. Thus, by way of example, in an aspect of the present disclosure, a drug for achieving the rapid depletion of LAA via the blocking of signal transduction for LAA may be a composition, molecule or drug including at least one of: a direct protein kinase C (PKC) inhibitor, e.g., GF109203X, and a direct protein kinase A (PKA) inhibitor, e.g., H-89. In another example, the PKC inhibitor may also include Ro-31-8220. As such, in another example, Ro-31-8220 may be used for the signal transduction blocking of LAA to achieve the rapid depletion of the LAA in a body of a patient (e.g., a human or animal patient). As such, a composition or molecule including at least one of: H-89, GF109203X, Ro-31-8220, or others, alone or in combination with others, may be used to block the signal transduction for LAA at a cellular level, thereby achieving the rapid depletion of the LAA to mitigate or reduce the growth of malignant tumor cells in accordance with one or more aspects of the present disclosure.

In another aspect of the present disclosure and as shown in FIGS. 7-10, in addition to the signal transduction blocker of LAA, it is also possible to use a transporter blocker of LAA to achieve the rapid depletion of LAA. That is, a transporter for LAA may be blocked, suppressed or inhibited to achieve the rapid depletion of LAA in malignant tumor or cancer cells. By way of example, a specific transporter for the LAA such as sodium-ascorbate co-transporters or sodium-dependent vitamin C transporters (SVCTs) may be inhibited or suppressed to achieve the rapid depletion of LAA, thereby mitigating or reducing the growth of malignant tumor or cancer cells.

It has been well-known that LAA may be accumulated in cells by two types of proteins: SVCTs and hexose transporters (GLUTs). SVCTs actively import ascorbate and are surface glycoproteins encoded by two different genes but having similar structure, namely, SVCT1 and SVCT2. SVCT1 is involved in whole-body homeostasis of LAA while SVCT2 protects metabolically active cells against oxidative stress. Further, SVCT2 is crucial for ascorbate uptake in metabolically active and specialized tissues, thus protecting them from oxidative stress (see Reference 51).

In other words, the transportation of LAA into cells are performed by two isoforms of SVCT transporters: namely, SVCT1 (hSVCT1) and SVCT2 (hSVCT2). LAA may be directly transported into cells via sodium-dependent LAA transporters, SVCT1 and/or SVCT2. Further, SVCT1 is predominantly expressed in epithelial cells, including those of intestine, kidney, skin and liver, and can transport greater amounts of LAA exceeding the internal requirement of these cells. SVCT2 may be localized in most tissues and SVCT2 may be expressed in lung and skeletal muscle as mentioned in an article titled “SVCT1 and SVCT2: key proteins for vitamin C uptake” by Savini, et al. (see Reference 51). As such, it is also possible to use SVCT2 blocker(s) as well as SVCT1 blockers, alone or in combination with each other to achieve the rapid depletion of LAA in malignant tumor or cancer cells to control their growth.

Additionally, as for transporters of LAA, another transporter of LAA is facilitative sodium-independent glucose transporters (GLUTs). Facilitative GLUT transporters can mediate absorption of dehydroascorbic acid (DHA), which is the oxidized form of LAA. However, most of LAA in blood exist in the reduced form which cannot be transported by GLUT, but rather by SVCTs. Further, there is a relatively much higher amount of glucose in blood, and LAA cannot compete against glucose for this transporter. Thus, for treatment of diabetes or supplemental support medicine, the blockade of the glucose transporter (SGLT2) has recently been approved by U.S. Food and Drug Administration (FDA). So far, more than four selective blockers of SGLT2 for the treatment of diabetes or supplemental support medicine have already been identified and used.

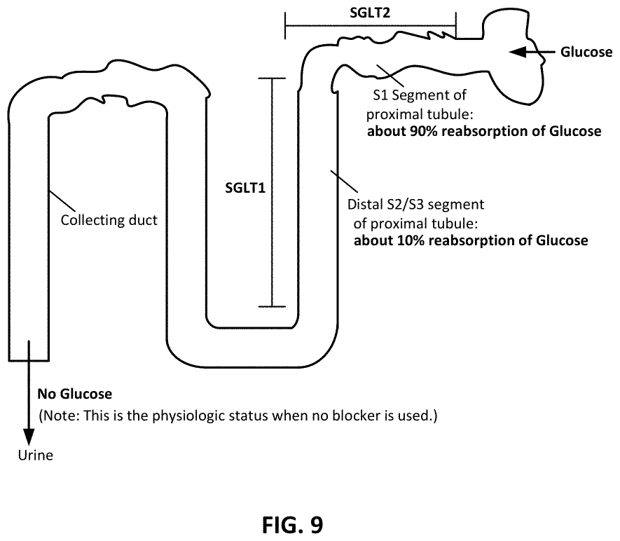

As an illustration for a better understanding of the use of a transporter blocker for LAA, in the context of SGLT2 blocker, FIG. 9 illustrates an example use of a transporter blocker for glucose. Gliflozins are sodium-glucose transport protein 2 (SGLT2) inhibitors which are prescription oral medications that inhibit reabsorption of glucose in the kidney and thus lower blood sugar. There are many brand and generic name drugs or combination products that contain SGLT2 inhibitors. As shown in FIG. 9, for glucose transporter, e.g., SGLT1 is present in epithelial systems including kidney tubules as well as gastrointestinal tract lining, while SGLT2 is present in almost all other cells in the body of a person. In short, when the use of SGLT2 inhibitors are taken by a patient it may result in a greater reduction of blood glucose levels, because about 90% reabsorption of glucose occurs by SGLT2 in S1 segment of proximal tubule and about 10% reabsorption of glucose occurs by SGLT1 in distal S2/S3 segment of proximal tubule. As a result, when the SGLT2 inhibitor drugs or combination products are taken by a patient, then reabsorption of glucose into blood may be cut by about 90% and thus the most of glucose will be eliminated via urine, thereby resulting in a significantly lower level of glucose in the blood of the patient. The efficacy of the drug may be dependent upon renal excretion and prevention of glucose from reentering the blood circulation by promoting glucosuria and the mechanism of action of Gliflozins is insulin independent.

Based on the above results, many SGLT2 inhibitors or combination products that contain SGLT2 inhibitors are available in the form of oral medications which manufactured and sold by major pharmaceutical companies, for example, Canagliflozin, Ertugliflozin, Empagliflozin, etc. Canagliflozin was the first SGLT2 inhibitor approved by the U.S. FDA in March of 2013. These Gliflozins are currently enjoying high popularity by both patients and physicians who treat diabetic patients, as they not only control blood glucose levels but also reduce body weight and heart attack.

Similarly, transporter blockers or inhibitors for LAA may be effectively used to achieve the rapid depletion of LAA in malignant tumor or cancer cells, thereby mitigating and reducing the growth of the malignant tumor or cancer cells in human patients and experimental animal models. This is because the malignant cells need LAA for their survival and growth, as mentioned earlier. As such, in an aspect of the present disclosure, when the transporters of LAA (e.g., SVCT1 and/or SVCT2) are blocked or inhibited, LAA becomes depleted, thereby having beneficial effects on the controlling the growth of malignant cells. In other words, the transporter inhibitors of LAA may be administered to a patient via compounds, molecules, drugs or other medicinal means that include one or more LAA transporter inhibitors to achieve the rapid depletion of LAA in the malignant tumor or cancer cells. In fact, the beneficial effects of LAA depletion (or low LAA) on cancer cure was demonstrated in cancer xenograft models in guinea pigs by using the very low amount of LAA or LAA-depleted diet, as shown in FIG. 5-6.

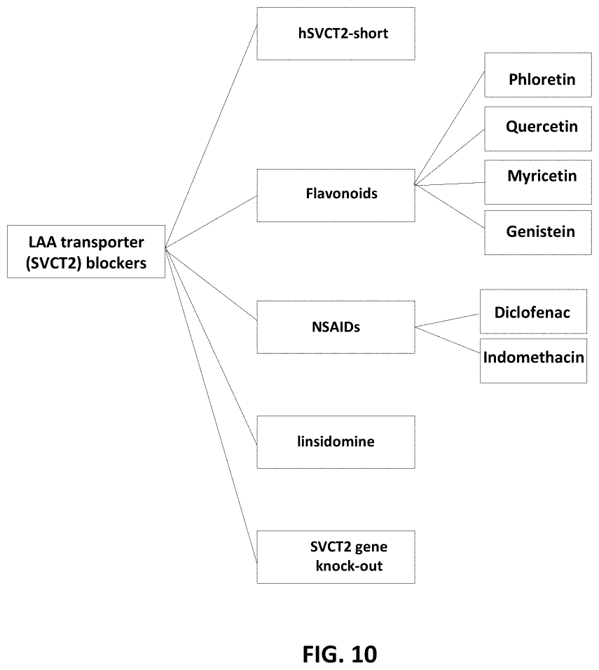

In accordance with an aspect of the present disclosure, by way of example, FIG. 10 illustrates example LAA transporter blockers in a summary form. As shown in FIG. 10, the transporter of LAA (e.g., SVCT2) for entry into tumor and/or cancer cells may be effectively blocked by using one or more of the following means: (1) expression of a short form of human SVCT2 (hSVCT2-short) in which 345 bp is deleted; (2) flavonoid phloretin, a known inhibitor of SVCT1 and/or SVCT2; (3) flavonoids such as quercetin, myricetin, and genistein, most of which are generally regarded safe by USA FDA and known inhibitors of SVCT1 and/or SVCT2 (see Reference 25); (4) non-steroidal anti-inflammatory drugs (NSAIDs) such as diclofenac and indomethacin; (5) an oxidant and NO-donor Sin-1, also known as linsidomine; and (6) knock-down or knock-out of SVCT2 using Lentivirus or CRISPR techniques. In other words, for the rapid depletion of LAA to control the growth of malignant tumor cells in a body of a patient (e.g., a human or animal patient), a pharmaceutical composition or drug including one or more of these molecules may be administered to the patient orally, intravenously or other methods. In one example, the advanced molecular biology techniques such as using lentivirus shRNA vectors and/or CRISPR techniques may be employed to achieve the rapid depletion of LAA, in addition to small molecule inhibitors as described herein, including naturally-occurring flavonoids such as phloretin, quercetin, genistein, and others. Further, the means for achieving the rapid depletion of LAA in malignant tumor or cancer cells may not be limited to these described herein, but may include various other techniques or means configured to achieve the effective blocking of the signal transduction for LAA as well as blocking of transporter of LAA, such as any presently known or future means.

In an aspect of the present disclosure, in one example, the short isoform or hSVCT-short may be used as the LAA transporter block because the hSVCT-short arises by alternative splicing and encodes a protein that strongly inhibited the function of SVCT2. In the example, the short form of human SVCT2 (e.g., hSVCT2-short) may be used to mitigate or eliminate the growth of malignant tumor cells via the rapid depletion of LAA in a patient. The short isoform or hSVCT2-short arises by alternative splicing and encodes a protein that strongly inhibited the function of SVCT2. As a result, hSVCT2-short may serve one of the effective LAA transporter blockers, as illustrated in part by a study entitled “A human sodium-dependent vitamin C transporter 2 isoform acts as a dominant negative inhibitor of ascorbic acid transport” by Eugene A. Lutsenko, et al. (see Reference 22), which is incorporated by reference herein in its entirety. As mentioned earlier, and according to the study above, the short isoform gives rise to a protein, i.e., hSVCT2-short protein, in which 345 bp is deleted without a frame shift, is unable to transport LAA. Further, in the short protein, transmembrane domains 5 and 6 are missing with a partially deleted domain 4. The short dominant-inactive protein (hSVCT2-short) arises by alternative splicing and encoding a protein that strongly inhibits the function of SVCT2. That is, sSVCT2-short is a short isoform that acts as a dominant-negative inhibitor of LAA transport through protein-protein interaction. As such, hSVCT2-short is one of the effective inhibitors of the transport of LAA and may be used as a means for achieving the rapid depletion of LAA, alone or in combination with other means described herein. In one application, a compound or drug including hSVCT2-short, alone or in combination with others disclosed herein, may be administered to the person either orally through medicinal tablets, caplets or capsules, or parenterally through intravenous infusion methods, for the purpose of achieving the rapid depletion of LAA in the body of the person, followed by the supplementation of a high dose LAA, in a cyclic administration protocol to mitigate or reduce the growth of malignant tumor cells.

In another aspect of the present disclosure, in one example, some of the known other inhibitors of SVCT1 and SVCT2, for example, flavonoids may be used. By way of example, flavonoid phloretin may be used, alone or in combination with others described herein, for achieving the rapid depletion of LAA in a person. Flavonoids are a diverse group of phytonutrients frequently found in many fruits and vegetables, which exhibit powerful antioxidants with anti-inflammatory and immune system benefits. A study titled “Sodium-dependent vitamin C transporter 2 (SVCT2) is necessary for the uptake of L-ascorbic acid into Schwann cells” by Burkhard Gess, et al. (see Reference 23), which is incorporated herein by reference in its entirely, shows that flavonoids have been shown to have inhibitory effect on glucose and LAA transport in vivo. In particular, in an aspect of the present disclosure, flavonoid phloretin, flavonoid quercetin, and/or flavonoid kaempferol may be used as the inhibitor of SVCT1 and SVCT2 for achieving the rapid depletion of LAA in the person. As such, in one application, a compound or drug including flavonoid phloretin, alone or in combination with others disclosed herein, may be administered to the person either orally through medicinal tablets, caplets or capsules, or parenterally through intravenous infusion methods, for the purpose of achieving the rapid depletion of LAA in the body of the person, in a cyclic administration protocol to mitigate or reduce the growth of malignant tumor cells.

In another aspect of the present disclosure, in one example, flavonoid quercetin, myricetin and/or genistein, alone or in combination with others disclosed herein, can be used as an inhibitor of SVCT1 and SGLT2 for the purpose of achieving the rapid depletion of LAA in a body of a patient (e.g., a human or animal patient). Their inhibitory effects of SVCT1 and SGLT2 also have been documented in another study entitled, “Flavonoid inhibition of sodium-dependent vitamin C transport (SVCT1) and glucose transport isoform 2 (SGLT2), intestinal transporters for vitamin C and glucose” by Jian Song, et al. (see Reference 25), which is incorporated herein by reference in its entirety. In the study, it was shown that when cells transfected with SVCT1(h) were incubated with ascorbate 10-400 uM with or without 50 uM quercetin (or myricetin or genistein), LAA transporter SVCT1(h) was inhibited about 80% by quercetin (see Reference 25).

Further, in another aspect of the present disclosure, flavonoids, in particular, quercetin, may be used to achieve the suppression of SVCT because the compounds can also inhibit the SVCT2 expressed in blood and/or bone marrow cells based on the structural similarities with many conserved amino acids between SVCT1 and SVCT2 and also, there is direct evidence for the inhibition of SVCT2 in cortical neurons and neuroblastoma cells. In one example, a journal article titled “The Na+-dependent L-ascorbic acid transporter SVCT2 expressed in brainstem cells, neurons, and neuroblastoma cells is inhibited by flavonoids” by Teresa Caprile, et al. (see Reference 27) reports that when cortical neurons or cerebellar neurons were incubated in the presence of 200 uM quercetin, the LAA transport SVCT2 was decreased by 80%. In another example, it reports that flavonoid phloretin shows similar inhibition effect of 60%-70% on SVCT2 when neurons isolated from brain cortex and cerebellum were incubated with phloretin (see Reference 27). Further, in long term toxicity experiments in animals such as rats and hamsters, it was shown that safe quercetin doses were at least 400 mg/kg/day and as high as 4000 mg/kg/day and as such, quercetin dose of 4 g may be administered to humans orally without side effects (see Reference 25).

As such, as mentioned above, flavonoid quercetin, phloretin, myricetin and/or genistein, alone or in combination with others disclosed herein, may be used as the LAA transport SVCT2/SVCT1 inhibitor to achieve the rapid depletion of LAA in a patient. Thus, in one application, a compound or drug including flavonoid quercetin, myricetin and/or genistein, alone or in combination with others disclosed herein, may be administered to the patient either orally through medicinal tablets, caplets or capsules, or parenterally through intravenous infusion methods, for the purpose of achieving the rapid depletion of LAA in the body of the patient, in a cyclic administration protocol to mitigate or reduce the growth of malignant tumor cells.

In another aspect of the present disclosure, certain nonsteroidal anti-inflammatory drugs (NSAIDs) such as indomethacin (e.g., Indocin™) or diclofenac may also be used to achieve the rapid depletion of LAA in a body of a patient (e.g., a human or animal patient). That is, certain NSAIDs such as indomethacin (e.g., Indocin™) or an over-the-counter medicine diclofenac (e.g., Cambia™, Cataflam™, Voltaren™, Zipsor™, etc.) inhibit the uptake of LAA by inhibiting SVCT2 in trophoblast cells as shown in a study titled “Expression and characterization of vitamin c transporter in the human trophoblast cell line HTR-8/SVneo: effect of steroids, flavonoids and NDAIDs” C. Biondi, et al. (see Reference 28), which is incorporated herein by reference in its entirety. Generally, indomethacin and diclofenac are NSAIDs that work by blocking the body's production of certain natural substances that cause inflammation. However, because of their inhibitory nature of the SVCT2, they may be used as the means for achieving the rapid depletion of LAA in the body of the person. Further, the study reports that 17β-estadiol, quercetin, genistein and diclofenac show the very strong inhibitory action for LAA transporter SVCT2 in HTR-8/SVneo cells (see Reference 28). As such, in one application, a compound or drug including indomethacin or diclofenac, alone or in combination with others disclosed herein, may be administered to the person either orally through medicinal tablets, caplets or capsules, or parenterally through intravenous infusion methods, for the purpose of achieving the rapid depletion of LAA in the body of the person, in a cyclic administration protocol to mitigate or reduce the growth of malignant tumor cells.

In another aspect of the present disclosure, in another example, an oxidant such as SIN-1 may be used to achieve the rapid depletion of LAA in a body of a patient (e.g., a human or animal patient). It is noted that the expression or function of the SVCT2 can be negatively modulated by the presence of an oxidant and NO-donor [3-(4-Morpholinyl) sydnonimine hydrochloride (SIN-1) or Linsidomine], as reported in a journal article, “Sodium-dependent vitamin c transporter SVCT2: expression and function in bone marrow stromal cells and in osteogenesis” by Sadanand Fulzele, et al. (see Reference 29) which is incorporated herein by reference in its entirety. Linsidomine is a vasodilator which is a metabolite of the anti-anginal drug molsidomine and acts by releasing NO from the endothelial cells non-enzymatically. As such, because of its negative modulation nature of SVCT2, the oxidant such as Sin-1 or Linsidomine may be used to achieve the rapid depletion of LAA in a body of a patient (e.g., a human or animal patient). By way of example, 600 uM of SIN-1 may cause up to 40% decrease in LAA uptake in the bone marrow stromal cells (BMSCs) (see Reference 29). As such, in one application, a compound or drug including SIN-1 or Linsidomine, alone or in combination with others disclosed herein, may be administered to the person either orally through medicinal tablets, caplets or capsules, or parenterally through intravenous infusion methods, for the purpose of achieving the rapid depletion of LAA in the body of the person, in a cyclic administration protocol of the rapid depletion of LAA and the supplementation of a high dose LAA to mitigate or reduce the growth of malignant tumor cells.

In another aspect of the present disclosure, in one example, a knock-down or knock-out of SVCT2 gene may be used to achieve the rapid depletion of LAA in a body of a patient (e.g., a human or animal patient). That is, it is also possible that SVCT2 gene may be knocked-down or knocked-out by using a specific interfering siRNA to SVCT2 or lentivirus shRNA. Knock-down by lentivirus shRNA followed by subsequent autophagy and apoptosis of bone marrow stromal cells have been reported. By way of example, the lentiviral particles shSVCT2, shControl, Polybrene® and puromycin may be used. BMSCs may be plated at 30%-50% confluence and transfected with appropriate dilutions of lentivirus particles and polybrene, and 48 hours after transfection, the cells may be cultured in growth medium containing puromycin (2 ug/ml) to harvest the transfected BMSC cells. The real-time PCT shows the efficiency of shRNA activity and uptake assay. In the case of osteogenesis, using a lentivirus-based shRNA knockdown resulted in the knockdown efficiency of lentivirus shSVCT2 in BMSCs of 50%-60% (see Reference 29), which shows significant inhibition of osteogenesis. Thus, by using the knock-down or knock-out of SVCT2 gene, the blocking of the transporter for LAA may be carried out. As such, in one application, a compound or drug including capability of knock-down or knock-out of SVCT2 gene, alone or in combination with others disclosed herein, may be administered to the person either orally through medicinal tablets, caplets or capsules, or parenterally through intravenous infusion methods, for the purpose of achieving the rapid depletion of LAA in the body of the person, in a cyclic administration protocol to mitigate or reduce the growth of malignant tumor cells.

Thus, in accordance with one or more aspects of the present disclosure, a combination of the blocking of the signal transduction of LAA and the blocking of the transporter of LAA may be employed at a molecular and/or cellular level, thereby resulting in the rapid depletion of LAA in a patient. This in turn may enable the provision of a repeated administration of alternation of the LAA depletion and LAA supplementation in a shortened cyclic fashion, as a therapeutic treatment for a certain type of disease or malignant tumor cells. That is, by undergoing a cyclic administration of rapid depletion of LAA followed by supplementation of high dose LAA or vice versa over a shortened period of time, patients with various types of malignancies (e.g., myeloma, acute myelogenous leukemia, myelodysplastic syndromes, acute lymphoblastic leukemia, sarcoma, neuroblastoma, breast cancer, testicular or prostate cancer, pancreatic cancer, non-small cell lung cancer, and advanced colorectal cancer, etc.) may receive benefits and be effectively treated or have their malignancies under control. It is also noted that SVCT2 is expressed in breast cancer, prostate cancer, and advanced colorectal cancer. Thus, through employing one or more aspects of the present disclosure, it is also expected that the present technology may be used to combat these types of cancers.

Further, various aspects of the present disclosure may be employed in conjunction with traditional treatment such as surgery, radiation, chemotherapeutic agents, heat treatment, etc. to enhance the therapeutic effect on patients. In other words, the novel treatment in accordance with the present disclosure, in particular, the manipulation of the rapid LAA depletion and the high-dose LAA supplementation in a cyclic fashion over a period of time may be used to further enhance the therapeutic efficacy of the traditional treatment. Further, the novel approach of the cyclic administration of LAA depletion and LAA supplementation may be used to enhance the therapeutic efficacy of either LAA depletion or LAA supplementation alone.

In another aspect of the present disclosure, the cyclic administration of the rapid LAA depletion and high dose LAA supplementation may be implemented with two-week interval, for example. That is, in one application example, the rapid depletion of LAA may be administered to a patent for two weeks and the high dose supplementation of LAA may be administered to the patient in next two weeks over a period of up to a six-month. The safety data on LAA depletion in diet for volunteers shows that patients may be placed on diet depleted of LAA up to six months (References 46 and 47). Also, cancer cells in our cell culture either grow and form visible colonies, or die out during 2 weeks period. As such, within a 6-month treatment period, the cyclic administration of rapid LAA depletion and high dose LAA supplementation may be designed and implemented for a patent and may be adjusted, monitoring the efficacy of the cyclic administration on target tumor cells in the patent.

In another aspect of the present disclosure, the cyclic administration of the rapid LAA depletion and high dose LAA supplementation may be implemented in conjunction with existing chemotherapy. By way of example, in another application example, the rapid depletion of LAA may be administered to a patient for two weeks and the high dose supplementation of LAA may then be administered to the patient in next two weeks and after that chemotherapy may be administered to the patient, repeating cyclic treatment over a period of time for up to a six-month. As such, within a 6-month treatment period, the cyclic administration of rapid LAA depletion, high dose LAA supplementation and chemotherapy may be designed and implemented for a patent and may be adjusted, monitoring the efficacy of the cyclic administration on target tumor cells in the patent.

As such, the present disclosure provides various novel methods and/or techniques for reducing or mitigating the growth of malignant tumor cells in a patient using the cyclic administration of the rapid depletion of LAA and the high dose LAA supplementation over a period of time as a standalone therapeutic treatment or in combination with other traditional therapeutic treatment. Further, in accordance with various aspects of the present disclosure, novel composition(s) or molecular drug(s) may be employed to achieve the rapid depletion of LAA in a controlled cyclic administration and manipulation of LAA to mitigate or reduce the growth of malignant tumor cells in a patient.

As used in the present disclosure, except explicitly noted otherwise, the term “comprise” and variations of the term, such as “comprising,” “comprises,” and “comprised” are not intended to exclude other additives, components, integers or steps.

The term “about” when used herein mean in the context of scalar value refers to ±10% of the scalar value. Also, the term “about” when used in the context of a range of values refers to a range that includes values from 10% lower than the lower value of the range to values 10% higher than the highest value of the range. The term “at least one of” followed by a list such as “a, b, c, and/or d” refers to a list comprising each member of the list, individually, or any combination of two or more members of the list, up to and including all members of the list and, optionally, including other elements not listed in the list.

The term “drug” or “active ingredient” refers to an agent, active ingredient compound or other substances, or compositions and mixture thereof that provide some pharmacological, often beneficial, effect. The term “dosage form” denotes any form of the formulation that contains an amount sufficient to achieve a therapeutic effect.

The terms “first,” “second,” and so forth used herein may be used to describe various components, but the components are not limited by the above terms. The above terms are used only to discriminate one component from other components, without departing from the scope of the present disclosure. Also, the term “and/or” used herein includes a combination of a plurality of associated items or any item of the plurality of associated items. A singular form may include a plural form if there is no clearly opposite meaning in the context. In the present disclosure, the term “include”, “exhibit”, or “have” used herein indicates that a feature, an operation, a component, a step, a number, a part or any combination thereof described herein is present. Further, the term “include”, “exhibit”, or “have” does not exclude a possibility of presence or addition of one or more other features, operations, components, steps, numbers, parts or combinations. Furthermore, the article “a” used herein is intended to include one or more items. Moreover, no element, act, step, or instructions used in the present disclosure should be construed as critical or essential to the present disclosure unless explicitly described as such in the present disclosure.

Although the present technology has been illustrated with specific examples described herein for purposes of describing example embodiments, it is appreciated by one skilled in the relevant art that a wide variety of alternate and/or equivalent implementations may be substituted for the specific examples shown and described without departing from the scope of the present disclosure. As such, the present disclosure is intended to cover any adaptations or variations of the examples and/or embodiments shown and described herein, without departing from the spirit and the technical scope of the present disclosure.

### REFERENCES

(1) Park, C H, Bergsagel, D E, McCulloch, E A: Ascorbic acid: A culture requirement for colony formation by mouse plasmacytoma cells. Science 174:720-722, 1971.
(2) Park, C H: Studies of growth characteristics of myeloma in vitro, Ph.D. Thesis, University of Toronto, 1971.
(3) Park, C H: The biological nature of the effect of ascorbic acid on growth of human leukemic cells. Cancer Res 45:3969-3973, 1985.
(4) Park C H, Kimler B F, Yi S Y, Park S H, Kim K, Jung C W, Kim S H, Lee E R, Rha M, Kim S, Park M H, Lee S J, Park H K, Lee M H, Yoon S S, Min Y H, Kim B S, Kim J-A and Kim W S: Depletion of L-Ascorbic Acid Alternating with its Supplementation in the Treatment of Patients with Acute Myeloid Leukemia or Myelodysplastic Syndromes. Eur J Haematol 83:108-118, 2009.
(5) Migliozzi, J A: Effect of Ascorbic Acid on Tumor Growth. Br. J. Cancer 35:448-453, 1979.
(6) Berger M D, Stinzing S, Yang D, Cao S, Miyamoto Y, Suenaga M, Soni S, Gopez R, Melendez E, Zhang W, Heinemann V, Lenz H.-J: Genetic Variations within the vitamin C transporter genes to predict outcome in metastatic colorectal cancer patients treated with first-line FO1FIRI and bevacezamab: Data from FIRE-3 trial. J Clin Oncol 35, 2017 (suppl; abstract 11507).
(7) Park C H, Kimler B F, Yi S Y, Park S H, Kim K, Jung C W, Kim S H, Lee E R, Rha M, Kim S, Park M H, Lee S J, Park H K, Lee M H, Yoon S S, Min Y H, Kim B S, Kim J-A and Kim W S: Depletion of L-Ascorbic Acid Alternating with its Supplementation in the Treatment of Patients with Acute Myeloid Leukemia or Myelodysplastic Syndromes. Eur J Haematol 83:108-118, 2009.
(8) Cheson B D, Bennett J M, Kopecky K J, et al. Revised recommendations of the international working group for diagnosis, standardization of response criteria, treatment outcomes, reporting standards for therapeutic trials in acute myeloid leukemia. J Clin Oncol 2003; 21:4642-9.
(9) Cheson B D, Bennett J M, Kantarjian H, et al. Report of international working group to standardize response criteria for myelodysplastic syndromes. Blood 2000; 96:3671-4.
(10) Smith B D, Levis M, Beran M, et al. Single-agent CEP-701, a novel FLT3 inhibitor, shows biological and clinical activity in patients with relapsed or refractory acute myeloid leukemia. Blood 2004; 103: 3669-76.
(11) https://articles.mercola.com/sites/articles/archive/2017/03/06/vitamin-c-cancer-treatment.aspx Vitamin C may be a potent adjunct to cancer treatment.
(12) Hoffer L J, Levine M, Assouline S, Melnychuk D, Padayatty S J, Rosadiuk K, Rousseau C, Robitaille L, Miller W H Jr. Phase I clinical trial of i.v. ascorbic acid in advanced malignancy. Ann Oncol 2008; 19:1969-74.
(13) Cameron E, Pauling L. Supplemental ascorbate in the supportive treatment of cancer: Prolongation of survival times in terminal human cancer. Proc Natl Acad Sci USA. 1976 October; 73(10):3685-9.
(14) Creagan E T, Moertel C G, O'Fallon J R, Schutt A J, O'Connell M J, Rubin J, Frytak S. Failure of high-dose vitamin C (ascorbic acid) therapy to benefit patients with advanced cancer. A controlled trial. N Engl J Med. 1979 Sep. 27; 301(13):687-90.
(15) Park, C H, et al., U.S. Pat. No. 6,989,143 B1.
(16) Park, C H, et al., EU Patent EU 1 278 536 B1.
(17) Moteki H., Shimamura Y, Kimura M and Ogihara M. Signal Transduction Pathway for L-ascorbic acid2 glucoside-induced DNA Synthesis and cell proliferation in primary cultures of rat hepatocytes. Eur J Pharmacol. May 15; 683 (1-3); 276-84, 2012.
(18) Moteki H, Kimura M, Sunaga K, Tsuda T, and Ogihara M. Signal transduction mechanism for potentiation by α1- and β2-adrenoceptor agonists of L-ascorbic acid-induced DNA synthesis and proliferation in primary cultures of adult rat hepatocytes. Eur J Pharmacol. 2013 Jan. 30; 700(1-3):2-12.
(19) Kimura M, Moteki H., Uchida M, Natsume H. Ogihara M: L-ascorbic acid- and L-ascorbic acid 2-glucoside accelerate in vivo liver regeneration and lower serum alanine aminotransaminase activity in 70% partially hepatectomized rats. Biol Pharm Bull. 2014; 37(4): 597-603.
(20) Tsukaguchi H, Toki T, Mackenzie B, Berger U B, Chen X Z, Wang Y, Brubaker R F, Hediger M A: A family of mammalian Na⁺-dependent L-ascorbic acid transporters. Nature 1999; 399, 70-75.
(21) Liang W-J, Johnson D, Jarvis S M: Vitamin C transport systems of mammalian cells. Mol Memb Biol. 2001, 18(1):87-95.
(22) Rivas C I, Zúñiga F A, Salas-Burgos A, Mardones L, Ormazabal V, Vera J C. Vitamin C transporters. J Physiol Biochem. 2008 December; 64(4):357-75.
(23) Lutsenko E A, Carcamo J M, Golde D W. A human sodium-dependent vitamin C transporter 2 isoform acts as a dominant-negative inhibitor of ascorbic acid transport. Mol. Cell Biol. April 2004, 24(8):3150-6.
(24) Gess B, Lohmann C, Halfter H, Young P. Sodium-dependent vitamin C transporter 2 (SVCT2) is necessary for the uptake of L-ascorbic acid into Schwann cells,” Glia, 2010 February 58(3):287-99.
(25) Song J, Kwon O, Chen S, Daruwala R, Eck P, Park J B, Levine M. Flavonoid inhibition of sodium-dependent vitamin C transporter 1 (SVCT1) and Glucose Transporter Isoform 2 (GLUT2), Intestinal Transporters for Vitamin C and Glucose. J Biol. Chem. Vol. 277, No. 18, May 2002, pp. 15252-15260.
(26) Park J B, Levine M: Intracellular accumulation of ascorbic acid is inhibited by flavonoids via blocking of dehydroascorbic acid and ascorbic acid uptakes in HL-60, U937 and Jurkat cells. J Nutr. 2000 May, 130(5):1297-302.
(27) Caprile T, Salazar K, Astuya A, Cisternas P, Silva-Alvarez C, Montecinos H, Milian C, de Los Angeles Garcia M, Nualart F. The Na+-dependent L-ascorbic acid transporter SVCT2 expressed in brainstem cells, neurons, and neuroblastoma cells is inhibited by flavonoids. J Neurochem. 2009 February; 108(3):563-77.
(28) Biondi C, Pavan B, Dalpiaz A, Medici S, Lunghi L, Vesce F. Expression and characterization of vitamin C transporter in the human trophoblast cell line HTR-8/SVneo: effect of steroids, flavonoids and NSAIDs. Mol Hum Reprod. 2007 January; 13(1):77-83.
(29) Fulzele S, Chothe P, Sangani R, Chutkan N, Hamrick M, Bhattacharyya M, Prasad P, Zakhary I, Bowser M, Isales C, Ganapathy V. Sodium-dependent Vitamin C Transporter SVCT2: Expression and Function in bone marrow stromal cells and in Osteogenesis. Stem Cell Res. 2013 Jan. 10(1):36-47.
(30) Sangani R, Periyasamy-Thandavan S, Pathania R, Ahmad S, Kutiyanawalla A, Kolhe R, Bhattacharyya A M, Chutkan N, Hunter M, Hill W D, Hamrick M, Isales C, Fulzele S. The crucial role of vitamin C and its transporter (SVCT2) in bone marrow stromal cell autophagy and apoptosis. Stem Cell Res. 2015 Sep. 15 (2):312-21.
(31) Hu X. CRISPR/Cas9 system and its applications in human hematopoietic cells. Blood Cells Mol Dis. 2016 November; 62:6-12.
(32) Tothova Z, Krill-Burger J M, Popova K D, Landers C C, Sievers Q L, Yudovich D, Belizaire R, Aster J C, Morgan E A, Tsherniak A, Ebert B L. Multiplex CRISPR/Cas9-Based Genome Editing in Human Hematopoietic Stem Cells Models Clonal Hematopoiesis and Myeloid Neoplasia. Cell Stem Cell. 2017 Oct. 5; 21(4):547-555.
(33) Hong S W, Lee S H, Moon J H, Hwang J J, Kim D E, Ko E, Kim H S, Cho I J, Kang J S, Kim D J, Kim J E, Shin J S, Jung D J, Jeong Y J, Cho B J, Kim T W, Lee J S, Kang J S, Hwang Y I, Noh D Y, Jin D H, Lee W J. SVCT-2 in breast cancer acts as an indicator for L-ascorbate treatment. Oncogene. 2013 Mar. 21; 32(12):1508-17.
(34) Keshari K R, Sai V, Wang Z J, Vanbrocklin H F, Kurhanewicz J, Wilson D M. Hyperpolarized [1-13C]dehydroascorbate MR spectroscopy in a murine model of prostate cancer: comparison with 18F-FDG PET. J Nucl Med. 2013 June; 54(6):922-8.
(35) Erichsen H C, Peters U, Eck P, Welch R, Schoen R E, Yeager M, Levine M, Hayes R B, Chanock S. Genetic variation in sodium-dependent vitamin C transporters SLC23A1 and SLC23A2 and risk of advanced colorectal adenoma. Nutr Cancer. 2008; 60(5):652-9.
(36) Tsao C S, Dunham W B, Leung P Y. In vivo antineoplastic activity of ascorbic acid for human mammary tumor. In Vivo. 1988 March-April; 2(2):147-50.
(37) Nunez Martin C, Ortiz de Apodaca y Ruiz A. Ascorbic acid in the plasma and blood cells of women with breast cancer. The effect of the consumption of food with an elevated content of this vitamin. Nutr Hopsp. 1995 November-December; 10(6):368-72. (article in Spanish).
(38) Qu J, Zhu X, Lu Y, Zhao C, Zhang H, Wang X, Gui X, Wang J, Zhang X, Zhang T, Pang C L K. The safety and pharmacokinetics of high dose intravenous ascorbic acid synergy with modulated electro-hyperthermia in Chinese patients with stage III-IV non-small cell lung cancer. Eur. J Pharma Sci. 2017; 109:412-418.
(39) Fischer A P, Miles S L. Ascorbic acid, but not dehydroascorbic acid increases vitamin C content to decrease Hypoxia Inducible Factor-1 alpha (HIF-1a) activity and reduce malignant potential human melanoma. Biomed. Pharmacother. 2017 86: 502-513.
(40) Chan S W and Reade P C. The role of ascorbic acid in oral cancer and carcinogenesis. Oral Dis. 1998 4(2):120-9;
(41) Cieslak J A, Cullen J J. Treatment of pancreatic cancer with pharmacological ascorbate. Curr Pharm Biotechnol. 2015; 18: 759-70.
(42) Cohen M, Bhagavan H N. Ascorbic acid and gastrointestinal cancer. J Am Coll Nutr. 1995 December; 14(6): 565-78.
(43) El Halabi I, Bejj any R, Nasr R, Mukherji D, Temraz S, Nassar F J, El Darsa H, Shamseddine A. Ascorbic acid in colon cancer: from the basic to clinical applications. Int. J. Mol. Sci. 2018 Sep. 13; 19(9): pii: E2752.
(44) Ngo B, Van Piper J M, Cantley L C, Yun J. Targeting cancer vulnerabilities with high-dose vitamin C. Nat Rev Cancer. 2019 May; 19(5):271-282.
(45) Mata A M, Mata A M, Carvalho R M, Alencar M V, Cavalcante A A, Silva B B. Ascorbic acid in the prevention and treatment of cancer. Rev Assoc Med Bras. 2016 62: 680-686.
(46) Crandon J H, Lund C C, Dill D B. Experimental Human Scurvy. New England J Medicine. 1940 Sep. 5; vol. 223, page 353-369.
(47) Medical Research Council. Vitamin-C Requirement of Human Adults (Experimental Study of vitamin-C deprivation in man). Lancet. Jun. 5, 1948; 853-858.
(48) Stenger M. Bipolar androgen therapy after progression on enzalutamide in metastatic castration-resistant prostate cancer. By Matthew Stenger Posted: Jan. 9, 2018 5:56:49 PM Last Updated: Jan. 9, 2018 5:56:49 PM Tweet this page.
(49) https://medicalxpress.com/news/2019-03-testosterone-prostate-cancer-recurrence-low-risk.html.
(50) Di Tano M, Raucci F, Vernieri C, Caffa I, Bueno R, Fanti M, Brandhorst S, Curigliano G, Nencioni A, de Braud F, Longo V D. Synergistic effect of fasting-mimicking diet and vitamin C against KRAS mutated cancers. Nature Communications 2020; 11:2332.
(51) Savini I, Rossi A, Pierro C, Avigliano L, Catani M V. SVCT1 and SVCT2: key proteins for vitamin C uptake. Amino Acids (2008) 34: 347-455.
(52) Migliozzi J A. Effect of Ascorbic Acid on Tumor Growth. Br. J Cancer 35:448-453, 1977.
(53) Telang S, Clem A L, Eaton J W, Chesney J. Depletion of Ascorbic Acid Restricts Angiogenesis and Retards Tumor Growth in a Mouse Model. Neoplasia 9:47-56, 2007.

What is claimed is:

1. A method for treating a patient with a disease, the method comprising administration of a repeated cycle of alternation of a rapid depletion of L-ascorbic acid (LAA) and supplementation of a high dose of LAA over a period of time,
wherein the rapid depletion of LAA is achieved by blocking of LAA signal transduction and/or by blocking of LAA transporter to control growth of malignant cells in a body of the patient.

2. The method of claim 1, wherein the rapid depletion of LAA or the supplementation of the high dose of LAA is done through an intravenous infusion method.

3. The method of claim 1, wherein the disease comprises myeloma, acute myelogenous leukemia, myelodysplastic syndromes, acute lymphoblastic leukemia, sarcoma, non-small cell lung cancer, melanoma, oral, gastrointestinal, colon, pancreatic, breast, or prostate cancer.

4. The method of claim 1, wherein the blocking of LAA signal transduction is performed by a composition or drug comprising at least one of: GF109203X or H-89.

5. The method of claim 1, wherein the blocking of LAA transporter is performed by composition or drug comprising at least one of: a short form of human SVCT2 (hSVCT2-short), flavonoid phloretin, quercetin, myricetin, genistein, a non-steroidal anti-inflammatory drug (NSAID) including indomethacin and/or diclofenac, or linsidomine.

6. A pharmaceutical composition for inducing a rapid depletion of L-ascorbic acid (LAA) in a body of a patient as a treatment of a disease,
wherein the pharmaceutical composition comprises a composition designed to perform at least one of: blocking of a LAA signal transduction and/or blocking of a LAA transporter; and
wherein the pharmaceutical composition is used as a depletion means in a cyclic administration of the rapid depletion of LAA and the supplementation of high dose LAA over a period of time.

7. The pharmaceutical composition of claim 6, wherein the disease comprises myeloma, acute myelogenous leukemia, myelodysplastic syndrome, acute lymphoblastic leukemia, sarcoma, non-small cell lung cancer, melanoma, oral, gastrointestinal, colon, pancreatic, breast, or prostate cancer.

8. The pharmaceutical composition of claim 6, wherein the composition comprises at least one of: GF109203X or H-89.

9. The pharmaceutical composition of claim 6, wherein the composition comprises at least one of: a short form of human SVCT2 (hSVCT2-short), flavonoid phloretin, quercetin, myricetin, genistein, a non-steroidal anti-inflammatory drug (NSAID) including indomethacin and/or diclofenac, or linsidomine.
